(19) United States
(12) Patent Application Publication
(10) Pub. No.: US 2025/0049404 A1
(43) Pub. Date: Feb. 13, 2025

(54) AUTOMATED ACTIVATION OF A HEATING FUNCTION OF AN X-RAY SOURCE OF AN X-RAY SYSTEM

(71) Applicant: Siemens Healthineers AG, Forchheim (DE)

(72) Inventor: Fabian SCHEIDIG, Baiersdorf (DE)

(73) Assignee: Siemens Healthineers AG, Forchheim (DE)

(21) Appl. No.: 18/795,544

(22) Filed: Aug. 6, 2024

(30) Foreign Application Priority Data
Aug. 9, 2023 (DE) 10 2023 207 636.5

(51) Int. Cl. A61B 6/00; A61B 6/10; A61B 6/58

(52) U.S. Cl. CPC A61B 6/4488; A61B 6/107; A61B 6/54; A61B 6/581

(57) ABSTRACT

A method for automated control of a heating function of an x-ray source of an x-ray system is described. In the method, it is automatically detected whether a preparatory action for a use of the x-ray system for a new patient is in progress and a heating operation for heating the x-ray source is started in the event that a preparatory action for a use of the x-ray system for a new patient has been detected. A control device for an x-ray system is also described. An x-ray system is further described.

### CROSS-REFERENCE TO RELATED APPLICATION(S)

The present application claims priority under 35 U.S.C. § 119 to German Patent Application No. 10 2023 207 636.5, filed Aug. 9, 2023, the entire contents of which is incorporated herein by reference.

### FIELD

One or more example embodiments relates to a method for automated control of a heating function of an x-ray source of an x-ray system. One or more example embodiments also relates to a control device for an x-ray system. One or more example embodiments further relates to an x-ray system.

### RELATED ART

Two- or three-dimensional image data that can be used for visualizing an imaged examination subject and in addition for further applications as well is routinely generated with the aid of modern imaging methods.

The imaging methods are often based on the detection of x-ray radiation. An x-ray imaging system comprises an x-ray source via which x-rays are emitted in the direction of an examination subject. The x-rays partially penetrate the examination subject and some of the radiation is absorbed by the examination subject. The transmitted fraction of the x-ray radiation is detected by an x-ray detector arranged opposite the x-ray source. The x-ray source has a component called an x-ray tube which must be constantly maintained at an operating temperature during operation.

In most situations, the heating coil of the x-ray tube is heated to the acquisition temperature essentially only for the duration of the x-ray acquisition in order not to subject it to stress unnecessarily. To ensure the desired acquisition temperature is reached as quickly as possible, the heating coil can be heated during a preheating phase via a heating current to an operating temperature which is dimensioned such that no tube current flows, in particular during the preheating phase.

For this purpose, the x-ray source comprises heating coils through which a heating current flows already before an actual operation of the x-ray system. In the switched-on state, an x-ray imaging system requires approximately 2 kW of electrical power on account of the heating process.

Approximately 4 seconds are required in order to preheat the x-ray source before an x-ray imaging system is ready for operation. In conventional practice, the x-ray source is heated constantly in order to avoid a delay due to this preheat time during patient turnarounds, though this leads to high energy consumption.

In addition to x-ray imaging systems, x-ray sources are also used for x-ray systems that provide a therapeutic function.

### SUMMARY

When x-ray systems of this type are in operation it is desirable to minimize their energy consumption and to maximize their availability.

One or more example embodiments achieves delay-free operation of an x-ray system while providing reduced energy consumption.

This is achieved via a method for automated control of a heating function of an x-ray source of an x-ray system according to claim 1, a control device for an x-ray system according to claim 11, and an x-ray system according to claim 12.

### BRIEF DESCRIPTION OF THE DRAWINGS

One or more example embodiments is explained with reference to the attached figures, in which:

FIG. 1 shows a flowchart illustrating a method for automated control of a heating function of an x-ray source of an x-ray system according to an exemplary embodiment of the invention,

FIG. 2 shows a schematic representation of a control device according to an exemplary embodiment of the invention,

FIG. 3 shows a schematic representation of an arrangement for examining patients via an x-ray system according to an exemplary embodiment of the invention.

### DETAILED DESCRIPTION

In the method according to one or more example embodiments for automated control of a heating function of an x-ray source of an x-ray system, it is automatically detected whether a system movement, in particular a preparatory operation or preparatory process, and preferably a preparatory action, is in progress for a use of the x-ray system for a new patient. A “new” patient is to be understood as a patient who approaches the x-ray system for the purpose of a treatment or examination via the x-ray system because previously they have not yet been presented for treatment or examination on or in the x-ray system. The automated detection of the system movement, in particular the preparatory action, is accomplished by deploying technical monitoring equipment for monitoring operations being conducted in the environment of the x-ray system which are indicative of an approach of a patient and/or of an imminent start of an operation of the x-ray system, or by an automatic communication with data processing devices which acquire and process data which is likewise correlated with an imminent examination or treatment and an imminent start of operation of the x-ray system. System movement is to be understood as a change in a subsystem of the overall configuration of the x-ray system or a change in the overall system which is related to a change in the operating mode of the x-ray system and preferably is in a causal relationship with the change in operating mode. Change is to be understood in particular as the changes relating to the above-described preparatory actions in connection with the operation or operating mode of the x-ray system. An operating mode comprises active modes in which an active operation is started or carried out or terminated, and a standby mode in which the x-ray system is simply held in readiness, in which event, according to one or more example embodiments, the heating operation is powered down or completely interrupted in this standby mode. A “preparatory action”, in this context, is intended to include an action by a person in order to prepare for an examination or treatment of a patient, though the term “preparatory action” is also intended to cover technical operations, such as, for example, the automated setting or adjustment of a technical function of the x-ray system to match a patient or an examination or treatment of a patient. An x-ray system is to be understood as a system for examining an examination subject or patient, preferably a human being or an animal, or for treating a patient, which system employs x-ray beams for this purpose. An x-ray system preferably comprises a technical function based on the emission of x-rays for the medical imaging of such a patient. Alternatively, an x-ray system may also comprise a technical function based on the emission of x-rays for the medical treatment of a patient. Advantageously, because the control of the x-ray source is coordinated with a preparatory action, the x-ray system must be heated only when an examination or a treatment of a patient is also being performed or about to be performed via the x-ray system. The power consumption of the heating operation can advantageously be reduced to a necessary minimum. On the other hand, the automated detection of the preparatory action and the automated starting of the heating operation prevent a delay occurring between examinations or treatments of different patients via the x-ray system, which delay could reduce the throughput rate per unit of time. A reduction in the utilization of the capacity of the x-ray system is accordingly prevented as a result of the approach according to one or more example embodiments. Consequently, an improvement in the resource efficiency of the operation of an x-ray system is achieved. In particular, cost savings can be made or a reduced cost-effectiveness due to a lower throughput rate prevented. In order to save further heating energy, the heating of the x-ray system can be terminated automatically following the completion of an imaging procedure or treatment via the x-ray system. The time period during which the x-ray system is heated is advantageously limited to a time interval in which the x-ray system is active, thus saving electrical energy for operating the x-ray system, in particular heating energy. Because the heating elements of the x-ray system are switched off intermittently, the heating elements, in particular the heating coils used for the heating operation, are exposed to less wear and tear and consequently their useful life can be extended.

The control device according to one or more example embodiments has a detection unit for automatically detecting whether a preparatory action for a use of an x-ray system for a new patient is in progress. Also part of the control device according to one or more example embodiments is a start unit for starting a heating operation for heating an x-ray source of the x-ray system in the event that a preparatory action for a use of the x-ray system for the new patient has been detected. The control device according to one or more example embodiments shares the advantages of the method according to one or more example embodiments for automated control of a heating function of an x-ray source of an x-ray system.

The x-ray system according to one or more example embodiments has an x-ray source for emitting x-rays and a heating element for heating the x-ray source. The x-ray system further comprises an x-ray detector for detecting x-rays emitted by the x-ray source. Also part of the x-ray system according to one or more example embodiments is a control device according to one or more example embodiments. The x-ray system according to one or more example embodiments shares the advantages of the control device according to one or more example embodiments.

Most of the aforementioned components of the control device according to one or more example embodiments can be realized wholly or partly in the form of software modules in a processor of a corresponding computing system, for example by a control device of an x-ray system or a computer that is used for controlling such a system. A largely software-based implementation has the advantage that computing systems already used previously in the prior art can also be easily upgraded via a software update in order to operate in the manner according to one or more example embodiments. In that respect the object is also achieved via a corresponding computer program product comprising a computer program which can be loaded directly into a memory unit of a control device of an x-ray system or of another computing system and has program sections for performing the steps of the method according to one or more example embodiments when the program is executed in the control device or the computing system. In addition to the computer program, such a computer program product may, where appropriate, comprise additional constituent parts, such as for example a set of documentation, and/or additional components, including hardware components, such as for example hardware keys (dongles, etc.), to enable use of the software.

A computer-readable medium, for example a memory stick, a hard disk drive or some other transportable or permanently installed data medium, on which the program sections of the computer program that can be read in and executed by a computing system are stored, can serve for transport to the computing system or to the control device and/or for storage on or in the computing system or the control device. For this purpose, the computing system can have for example one or more cooperating microprocessors or the like.

The dependent claims as well as the following description in each case contain particularly advantageous embodiments and developments of the invention. In this regard, in particular the claims of one claims category may also be developed analogously to the dependent claims pertaining to a different claims category. Furthermore, the different features of different exemplary embodiments and claims may also be combined within the scope of the invention in order to form new exemplary embodiments.

A start time for a heating operation for heating the x-ray source is preferably specified in the event that a preparatory action for a use of the x-ray system for a new patient has been detected. Finally, the heating operation is started at the specified start time. In the simplest case, after a preparatory action has been detected, the heating operation is started immediately. If a variety of information is available in relation to the preparatory action and the minimum required time until the commencement of the examination or the medical treatment, this information as well as the preheating time already described above, preferably at least 4 seconds, can be used as a basis for specifying the time for the starting of the heating operation.

Advantageously, the energy consumption for the heating of the x-ray source can be further optimized or in particular minimized via an adjusted start time for the heating operation of the x-ray source.

In a variant of the method according to one or more example embodiments for automated control of a heating function of an x-ray source of an x-ray system, the automated detection comprises identifying a type of the detected preparatory action and the start time of the heating operation is specified as a function of the type of preparatory action. The minimum required time up to the commencement of an examination or treatment of a patient can be deduced on the basis of the established type of preparatory action. In particular, there exists an order of different preparatory actions which are carried out sequentially. Based on the identified type of preparatory action and the knowledge of the minimum duration of the individual sequentially running preparatory actions, it is therefore possible to calculate the minimum time required for all of the preparatory actions that are still to be performed, and in particular the minimum time still required at the time of performing a specific type of preparatory action up to the commencement of the examination or treatment. Advantageously, a start time of a heating operation for heating the x-ray source can be determined particularly precisely, as a result of which a particularly large amount of energy can be saved and the throughput rate of a use of an x-ray system can be optimized.

In detail, the detected preparatory action preferably comprises an operation for transferring the patient to the x-ray system. In particular, the patient transfer operation can be monitored by automatically and the position of the patient can be determined on the basis of this monitoring data. In most cases it is usual for a patient to take a predetermined route to the x-ray system. If a moving object is automatically detected on this route, it can be inferred that a patient is approaching the x-ray system. This approach action can advantageously be interpreted as a sign for an examination or treatment procedure that is about to be started and the heating operation of the x-ray source of the x-ray system can be started in timely fashion.

The operation for transferring the patient particularly preferably comprises an opening of a door to a shielded radiology room in which the x-ray system is installed. The x-ray system is located in a shielded radiology room in order to shield the environment and in particular medical staff against x-ray radiation. The patient has to open the door of the shielded radiology room in order to gain access to the x-ray system. Depending on the time that the door of the shielded radiology room is opened, an earliest time for a start of an examination or treatment can be predicted and consequently also a time for a starting of the heating operation of the x-ray source can be determined, where the time of starting the heating operation of the x-ray source must precede the time of the commencement of the examination or treatment by the heat-up time of the x-ray source, preferably at least 4 seconds.

In the above-described monitoring operation, the opening of the door is preferably detected via sensors. Advantageously, an entering of the shielded radiology room can be detected via the sensor-based detection of a movement of the door, in particular an opening of the door. A suitable sensor for this preferably comprises a contact sensor, in which case a breaking of contact between door and parts of the doorframe can be detected when a door is opened. A position of a patient in the door region can advantageously be determined particularly easily and with little technical overhead.

The preparatory action preferably comprises an operation for adjusting a position of the patient for an x-ray imaging session and/or an x-ray treatment. Immediately prior to the start of an examination or treatment, a position of a patient relative to the x-ray detector and the x-ray source must be adjusted so that a predetermined region of the patient, also referred to as the “region of interest”, is correctly recorded or treated. The adjustment may comprise the moving or the repositioning of a system composed of the x-ray detector and the x-ray source; it may also comprise the positioning of a patient couch on which the patient is already situated. With the adjustment of a position of the patient, an operation taking place immediately prior to the commencement of a treatment or examination can advantageously be detected automatically, thus enabling a start of the treatment or examination to be predetermined particularly precisely and consequently the start of the heating of the x-ray source to be specified particularly precisely also.

The detection of a preparatory action likewise preferably comprises an operation for registering the patient and/or for entering patient information. With this variant, there is advantageously no need for a patient to be monitored or detected via additional sensors since the patient registration system automatically makes data relating to a newly admitted patient available, on the basis of which data a time can be specified for a start of a heating operation of an x-ray source of an x-ray system.

In addition, a termination operation to conclude a treatment or an imaging procedure of the patient is preferably detected automatically, the heating operation for heating the x-ray source being terminated automatically on the basis of the detection of the termination operation. As a result of the automated termination of the heating operation, heating energy can advantageously be saved and the x-ray system can be maintained in a kind of standby state until the arrival of a new patient is detected.

The automated termination of the heating operation likewise preferably takes place only when a system movement or preparatory action has already been detected (not already at the time of the detection of the termination operation) which is indicative of the approaching of a new patient and/or an immediate or pending further operation of the x-ray system for a new examination or treatment. This system movement or preparatory action comprises in particular the already mentioned operation types:
an approaching of a patient,
an opening, by the patient, of a door to the room in which the x-ray system is installed,
a patient sign-in operation,
an adjustment operation for adapting the x-ray system to match a new patient.

The heating of the x-ray system is advantageously switched off only when the x-ray system is not required immediately afterward or alternatively promptly so that no delay to clinical operation is caused as a result of the deactivation of the heating of the x-ray system. The energy saving can advantageously be achieved without any reduction in the patient throughput rate of the x-ray system.

The termination operation particularly preferably comprises one of the following operation types:
an exiting of the x-ray system by the patient,
an opening, by the patient, of a door to the room in which the x-ray system is installed after the patient exits the x-ray system,
a patient sign-out operation.

The heating operation can advantageously be terminated close in time to the termination of an examination or a treatment, thereby saving heating energy.

The control device according to one or more example embodiments has a specification unit for specifying a start time of a heating operation for heating the x-ray source in the event that a preparatory action for a use of the x-ray system for a new patient has been detected. An operating time for a heating of an x-ray source can advantageously be further optimized via the specification unit and its function for specifying a start time and the energy consumption minimized accordingly during the operation of an x-ray system.

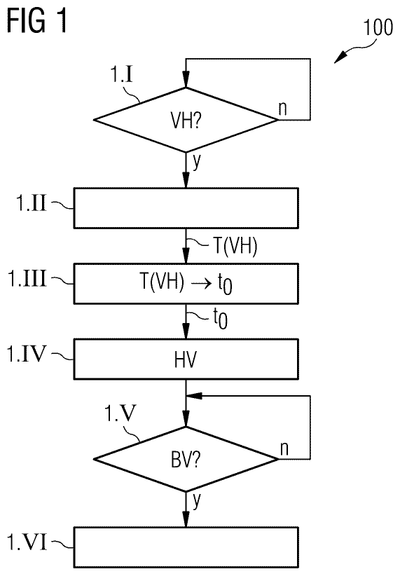

FIG. 1 shows a flowchart 100 illustrating a method for automated control of a heating function of an x-ray source of an x-ray system according to an exemplary embodiment of the invention.

At step 1.I, it is automatically detected whether a preparatory action VH for a use of the x-ray system for a new patient is in progress. In the event that such a preparatory action VH is detected, as indicated by “y” in FIG. 1, a transition is made to step 1.II. If no preparatory action VH is detected, as indicated by “n” in FIG. 1, step 1.I is repeated.

At step 1.II, it is determined which type T(VH) of preparatory action VH has been detected. The type T(VH) of preparatory action VH can be established for example on the basis of the type of information detected, for example sensor data from a sensor of a specific type or registration data RD from a patient registration computer.

If the type T(VH) of preparatory action VH has been identified, a start time t0 of a heating operation HV for heating the x-ray source is determined at step 1.III. In this case the start time t0 is also dependent on the type T(VH) of preparatory action VH. For example, a time interval between a registration of a patient and the start of an examination is longer than a time interval between the opening of a door to a shielded radiology room and the start of an examination. On the other hand, a time interval between the opening of a door of a shielded radiology room and the start of an examination is longer than a time interval between an adjustment of a patient position in an x-ray system and the start of an examination. The specified start time t0 is therefore dependent on the order of the possible preparatory actions or the position of the observed preparatory action in the order of the possible preparatory actions.

At step 1.IV, a heating operation HV is started at the specified start time t0.

At step 1.V, it is detected whether a termination operation BV to conclude a treatment or an imaging procedure of the patient is taking place.

In the event that a termination operation BV was detected at step 1.V, as indicated by “y” in FIG. 1, a transition is made to step 1.VI. In the event that no termination operation BV was detected at step 1.V, as indicated by “n” in FIG. 1, step 1.V is repeated.

At step 1.VI, the heating operation HV for heating the x-ray source is terminated automatically on the basis of the detection of the termination operation BV.

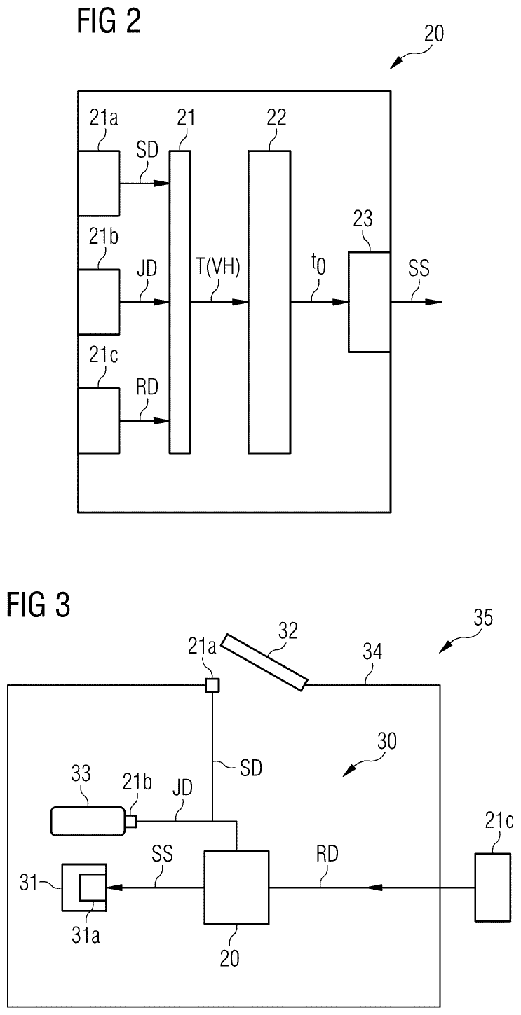

FIG. 2 shows a schematic representation of a control device 20 for an x-ray system 30 (see FIG. 3) according to an exemplary embodiment of the invention.

Part of the control device 20 is formed by a plurality of information gathering units 21a, 21b, 21c, which collect information SD, JD, RD that is forwarded to a detection unit 21 which is likewise part of the control device 20.

The information gathering units 21a, 21b, 21c comprise a door sensor 21a, which is configured to detect an opening of a door 32 (see FIG. 3) of a shielded radiology room 34 (see FIG. 3) in which the x-ray system 30 (see FIG. 3) is located and to forward sensor data SD to the detection unit 21. The information gathering units 21a, 21b, 21c also comprise an adjustment sensor 21b, which is configured to detect an adjustment of a patient position on a patient couch 33 (see FIG. 3) of the x-ray system 30 or to forward adjustment data JD to the detection unit 21. The information gathering units 21a, 21b, 21c additionally comprise a computer 21c, which is configured to receive registration data RD of a patient that is to be examined or treated via the x-ray system 30 and to forward said data to the detection unit 21. The detection unit 21 is configured to detect which type T(HV) of preparatory action is in progress on the basis of this information SD, JD, RD.

Also part of the control device 20 is a specification unit 22, which is configured to specify a start time t0 of a heating operation HV for heating an x-ray source of the x-ray system 30 on the basis of the detected type T(HV).

The control device 20 further comprises a start unit 23 for starting the heating operation HV at the specified start time t0. The control device 20 is configured to transmit a control signal SS to the x-ray source of the x-ray system 30 for the purpose of starting a heating operation HV.

FIG. 3 shows a schematic representation of an arrangement 35 for examining patients via an x-ray system 30 according to an exemplary embodiment of the invention.

The x-ray system 30 is installed in a shielded radiology room 34 which is accessible via a door 32. A patient that is to be examined (not shown) is first registered via a computer 21c and makes their way via the door 32 into the interior of the shielded radiology room 34 to the x-ray system 30. The x-ray system 30 has the control device 20 illustrated in FIG. 2 as well as an x-ray source 31 for emitting x-rays which has a heating element 31a for heating up the x-ray source 31. A patient couch 33 is also part of the x-ray system 30. A door sensor 21a is mounted on the door 32 and transmits sensor data SD to the control device 20 when the door is opened. A position sensor 21b is likewise arranged on the patient couch 33 in order to detect a change in position or an adjustment to a position of a patient and to transmit adjustment data JD to the control device 20. A data connection also exists between the computer 21c arranged outside of the shielded radiology room 34 and the control device 20 in order to communicate registration data RD to the control device 20. As already explained in connection with FIG. 2, the control device 20 transmits control data SS to the heating element 31a in order to start or terminate a heating operation, depending on which information is transmitted to the control device 20.

In conclusion, it is pointed out once again that the methods and structures described in detail in the foregoing are exemplary embodiments and that the basic principle may be varied in the most diverse ways by the person skilled in the art without leaving the scope of the invention insofar as it is set forth by the claims. It is also pointed out for the sake of completeness that the use of the indefinite articles “a” or “an” does not exclude the possibility that the features in question may also be present more than once. Similarly, the term “unit” does not rule out the possibility that said unit consists of a plurality of components which if necessary may also be distributed in space. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for automated control of a heating function of an x-ray source of an x-ray system, the method comprising:
automatically detecting whether a preparatory action for a use of the x-ray system for a new patient is in progress; and
starting a heating operation for heating the x-ray source in response to the preparatory action for using the x-ray system for the new patient being detected.

2. The method as of claim 1, wherein initially a start time for the heating operation for heating the x-ray source is specified in response to the preparatory action for the use of the x-ray system for the new patient being detected and the heating operation is started at the specified start time.

3. The method of claim 2, wherein
the automated detection comprises determining a type of the detected preparatory action, and
the start time of the heating operation is specified as a function of the type (of preparatory action.

4. The method of claim 1, wherein the preparatory action comprises an operation for transferring the new patient to the x-ray system.

5. The method of claim 4, wherein the operation for transferring the new patient comprises opening a door to a shielded radiology room in which the x-ray system is installed.

6. The method of claim 5, wherein the opening of the door is detected via sensors.

7. The method of claim 1, wherein the preparatory action comprises an operation for adjusting a position of the new patient for at least one of an x-ray imaging session or an x-ray treatment.

8. The method of claim 1, wherein the preparatory action comprises an operation for at least one of registering the new patient or for entering patient information.

9. The method of claim 1, wherein a termination operation to conclude a treatment or an imaging procedure of the new patient is detected automatically and the heating operation for heating the x-ray source is terminated automatically based on the detection of the termination operation.

10. The method of claim 9, wherein the termination operation comprises one of the following operation types:
an exiting from the x-ray system,
an opening of a door of a shielded radiology room in which the x-ray system is installed following the exiting from the x-ray system, or
a patient sign-out operation.

11. A control device, comprising:
a detection unit configured to automatically detect whether a preparatory action for a use of an x-ray system for a new patient is in progress; and
a start unit configured to start a heating operation for heating an x-ray source of the x-ray system in response to the preparatory action for the use of the x-ray system for the new patient being detected.

12. An x-ray system, comprising:
an x-ray source configured to emit x-rays;
a heating element configured to heat the x-ray source;
an x-ray detector unit configured to detect the emitted x-rays; and
the control device of claim 11.

13. The x-ray system of claim 12, wherein the x-ray system comprises one of the following system types:
an x-ray imaging system; or
an x-ray radiotherapy system.

14. A non-transitory computer program product comprising a computer program which, when executed by a control device of an x-ray system, causes the x-ray system to perform the method of claim 1.

15. A non-transitory computer-readable medium comprising program sections which, when executed by a computer unit, causes the computer unit to perform the method of claim 1.

16. The method of claim 3, wherein the preparatory action comprises an operation for transferring the new patient to the x-ray system.

17. The method of claim 2, wherein the preparatory action comprises an operation for adjusting a position of the new patient for at least one of an x-ray imaging session or an x-ray treatment.

18. The method of claim 2, wherein the preparatory action comprises an operation for at least one of registering the new patient or for entering patient information.

19. The method of claim 2, wherein a termination operation to conclude a treatment or an imaging procedure of the new patient is detected automatically and the heating operation for heating the x-ray source is terminated automatically based on the detection of the termination operation.
